(19) United States
(12) Patent Application Publication
(10) Pub. No.: US 2012/0041066 A1
(43) Pub. Date: Feb. 16, 2012

(54) MEDICAL FOODS FOR THE TREATMENT OF DEVELOPMENTALLY-BASED NEUROPSYCHIATRIC DISORDERS VIA MODULATION OF BRAIN GLYCINE AND GLUTATHIONE PATHWAYS

(76) Inventor: Jay L. Lombard, New City, NY (US)

(21) Appl. No.: 13/210,808

(22) Filed: Aug. 16, 2011

Related U.S. Application Data
(60) Provisional application No. 61/374,225, filed on Aug. 16, 2010.

(51) Int. Cl. A61K 31/197; A61P 25/00; A61P 25/18

(52) U.S. Cl. 514/562

(57) ABSTRACT

Describe herein are medical foods, pharmaceutical compositions, methods of compounding them, and method of using them for the treatment of developmentally-based neuropsychiatric disorders including particularly autism, ADHD, and persistent developmental disorders. The medical foods and pharmaceutical compositions typically include a methylglycine compound or precursor compound and an acetylcysteine compound or precursor compound. These methylglycine and acetylcysteine compounds may be prepared for sustained release or delivery. In some variations, a method of treating a developmentally-based neuropsychiatric disorder includes first determining if a patient is at risk for such a disorder by examining either or both phenotypical and genotypical biomarkers. The biomarkers may be used to tailor the dose to be delivered by the medial food or pharmaceutical composition.

### CROSS REFERENCE TO RELATED APPLICATIONS

This patent application claims priority to U.S. provisional patent application Ser. No. 61/374,225, field on Aug. 16, 2010, titled “MEDICAL FOODS FOR THE TREATMENT OF DEVELOPMENTALLY-BASED NEUROPSYCHIATRIC DISORDERS VIA MODULATION OF BRAIN GLYCINE AND GLUTATHIONE PATHWAYS.”

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

The compounds and methods described herein related generally to medical foods, methods of making and using them, and/or compounds for the treatment of neurodevelopmentally based disorders, which may include, but not limited to, autism, PDD, childhood psychosis and schizophrenia.

### BACKGROUND

Developmentally-based neuropsychiatric disorders such as autism, schizophrenia, ADHD, and cognitive developmental delays are both difficult to diagnose early and difficult to treat. However, there is a strong motivation to diagnose early, at preclinical or prodromal stages of the pediatric neuropsychiatric disorder, since early intervention may blunt, reduce or even prevent the full expression of these disorders.

For example, autism is a complex developmental disability that interferes with, among other things, the normal development of the brain in the areas of social interaction and communication skills. It typically appears during the first three years of life and is the result of a neurological disorder which affects the functioning of the brain. Typically, autistic children and adults have difficulties in verbal and non-verbal communication, social interactions, and leisure or play activities.

According to the Autism Society of America (hereinafter the “ASA”), autism is generally characterized as one of five disorders coming under the umbrella of Pervasive Developmental Disorders (PDD), a category of neurological disorders characterized by severe and pervasive impairment in several areas of development, including social interaction and communications skills (DSM-IV-TR). The five disorders under PDD are Autistic Disorder, Asperger's Disorder, Childhood Disintegrative Disorder (CDD), Rett's Disorder, and PDD-Not Otherwise Specified (PDD-NOS). Specific diagnostic criteria for each of these disorders can be found in the Diagnostic & Statistical Manual of Mental Disorders (DSM-IV-TR) as distributed by the American Psychiatric Association (APA).

The most common of the Pervasive Developmental Disorders, autism affects an estimated 1 in approximately 200 births. Indeed, as of 2003-2004, as many as 1.5 million Americans are believed to have some form of autism. Such a number is on the rise inasmuch as, based on statistics from the U.S. Department of Education and other governmental agencies, autism is growing at a rate of 10-17 percent per year. At these rates, the ASA estimates that the prevalence of autism could easily reach 4 million Americans in the next decade.

Although autism is defined by a certain set of behaviors, it is a spectrum disorder in that its symptoms and characteristics can be present in a wide variety of combinations, from mild to severe. Therefore, autistic children and adults can exhibit any combination of the behaviors in any degree of severity. Two individuals, both with the same diagnosis, may have varying skills and display very different actions.

Although there is no known single known cause for autism, it is generally accepted that it is caused by abnormalities in brain structure or function. The theory of a genetic basis of the disorder is supported by the fact that, in many families, there appears to be a pattern of autism or related disabilities. While no one single gene has been identified as causing autism, researchers are searching for irregular segments of genetic code or clusters of genes that autistic children may have inherited. While researchers have not yet identified a single “trigger” that causes autism to develop, it also appears that some children are born with a susceptibility to autism.

It is possible that under certain conditions, a cluster of unstable genes may interfere with brain development resulting in autism.

We herein propose that candidate genes involved in the pathogenesis of autism may include those particularly related to the adverse effects of oxidative stress and inflammatory pathways on brain development. Genes primarily relevant to these conditions include those related to glycine and glutathione pathways. Impairments in these genes and/or the metabolic pathways related to these compounds leads to pathological consequences involving excitatory brain neurotransmitter receptors; including NMDA, AMPA and Nicotinic acetylcholine receptor subtypes. Further, the abnormality in the function of these receptors may be secondary to reduced antioxidant potential in the brain. Glycine metabolism in the brain plays a critical role in mitochondrial function, brain glutathione production and Alpha 4beta2 and NMDA receptor activity. Glycine function in the developing brain and the metabolic consequences of abnormal glycine metabolism are relevant to this discovery. Glycine acts as a precursor for serine, which functions as a co agonist of NMDA receptors. Glycine, through its conversion to serine, serves as a primary donor to cysteine pools. Thus, the identification of disturbances in brain glycine metabolism, as well as its remediation by molecular signals downstream of glycine and serine metabolism, are relevant to this invention.

Abnormal genes of oxidative stress pathways and increased oxidative stress have been reported in autism spectrum disorders. Polymorphisms of genes involved in glutathione metabolism, e.g. GSTP1 and GSTM1 are reportedly associated with autistic disorder. GPX1 GCG repeat and other gene polymorphisms such as the MnSOD ALA16 or the GPX1 Pro198Leu polymorphism, genes which mediate endogenous anti oxidant pathways, have been reported in autism.

Furthermore, oxidized mitochondrial proteins are markedly increased in autism and altered Ca(2+) homeostasis play a key interactive role in the cascade of signaling events leading to autism: plasma biomarkers of oxidative stress have been reported in autistic children and intracellular redox status GSH/GSSG redox ratio is decreased and percentage oxidized glutathione increased in both cytosol and mitochondria in the autism.

Recent genetic studies have implicated a number of candidate genes in the pathogenesis of Autism Spectrum Disorder (ASD), which similar to schizophrenia, involve interactions between neuregulin (NRG1) and nACHr receptors and glutamate receptors. These receptor subtypes are particularly critical to interactions between cognitive and emotional processes. Alpha4beta2 nAChRs and neurexin-1beta are coexpressed in hippocampal neurons, interestingly, human neurexin-1 gene dysfunctions have been implicated in nicotine dependence and in autism spectrum disorders. Dysfunctional neurexins, through downstream effects on alpha4beta2, may contribute to the etiology of autism.

Alpha4 and beta2 protein expression and receptor binding density as well as alpha4 mRNA levels are lower in parietal cortex in autism, while alpha7 did not change for any of these parameters. The data obtained, using complementary measures of receptor expression, indicate that reduced gene expression of the alpha4beta2 nicotinic receptor in the cerebral cortex is a major feature of the neurochemical pathology of autism, whilst post-transcriptional abnormalities of both this and the alpha7 subtype are apparent in the cerebellum. The findings point to dendritic and/or synaptic nicotinic receptor abnormalities that may relate to disruptions in cerebral circuitry development. The reported abnormalities in these receptor subtypes during brain development likely involves abnormal signaling related to axonal migration in these disorders. The migrational defects which characterize the neuropathological changes in autism and schizophrenia result in impaired cortical- subcortical-hippocampal communication networks. While the primary mechanisms involved in these migrational abnormalities are not completely understood, it is currently proposed that epigenetic and epistatic factors are critical to the emergence of these impairments. Thus, novel treatments directed at reducing the impairment at these developmentally related pathways is greatly needed in the field.

Over the past 35 years, the most widely studied psychopharmacologic agents in autism have been anti-psychotic medications. Originally developed for treating schizophrenia, these drugs have been found to decrease hyperactivity, stereotypic behaviors, withdrawal and aggression in autistic children. Four that have been approved by the FDA are clozapine (Clozaril), risperidone (Risperdal), olanzapine (Zyprexa) and quetiapine (Seroquel). However, only risperidone has been investigated in a controlled study of adults with autism. Unfortunately, like the antidepressants, these drugs all have adverse side effects, including, but not limited to, sedation.

As mentioned briefly above, a primary need in the field of schizophrenia, autism and pediatric psychotic disorders disease is the identification of etiologically significant biomarkers. Such identification, especially in preclinical or prodromal stages of these disorders, may provide a novel opportunity to reduce the probability of the full expression of these conditions, which if left untreated, almost invariably become chronic. It would clearly be desirable to identify a diagnostic tool for schizophrenia, autism and psychotic disorders of childhood that are highly specific, and highly sensitive. However, in the absence of such a marker, the identification of factors associated with a higher probability of developing such a condition may be acceptable if the clinical response possesses a significantly lower risk to a child than a conventional medication. Of importance the marker should signify the disease early in its course, as there is evidence that delays in diagnosis and intervention lead to a poorer prognosis. In addition, a method that is cost-effective and non-invasive would be of added value. Given that subclinical or pre-clinical psychotic disorders may predict proneness, intervention in at risk individuals holds the promise of better outcomes.

Thus, there is a need for compositions, such as particularly medical food and pharmaceutical compositions, which are effective for treatment of developmentally-based neuropsychiatric disorders such as autism. In particular, it would be useful to provide such compositions to at-risk patients, where risk is determined by one or more biomarkers indicating a susceptibility to such neuropsychiatric disorders. Described herein are candidate biomarkers and associated compositions (including medical foods and pharmaceutical compositions) that may be used to treat or prevent developmentally-based neuropsychiatric disorders, as well as systems and methods for determining if a patient is in need of such treatment.

### SUMMARY

Described herein are compounds for the treatment of developmentally-based neuropsychiatric disorders (such as autism) that typically include a methylglycine compound and an acetylcysteine compound. Also described are methods of compounding medical foods or pharmaceuticals for treatment of developmentally-based neuropsychiatric disorders and methods of treating a developmentally-based neuropsychiatric disorder using these medical foods or pharmaceutical compounds. These interventions are based on overcoming the adverse effects of glycine and serine metabolic disturbances through the unique combination of N acetylcysteine and sarcosine. While it is well known to those experienced in the field that both glycine and serine are ineffective in the amelioration of the molecular disturbances in schizophrenia and autism, it is a novel discovery that these so identified metabolic disturbances may be overcome via the administration of down stream amino acids. Thus, while serine is unable to function as a glutathione precursor and NMDA co agonist directly, the utilization of N acetylcysteine and sarcosine may be able to do so. Further, the methods and compositions described herein relate in general to a method of identifying phenotypical and genotypical biomarkers in preclinical or prodromal stages of a pediatric neuropsychiatric disorder and subsequently addressing the risk by potentially inhibiting the clinical expression of said disorder through the employment of a safe medical food compound.

### BRIEF DESCRIPTION OF THE DRAWINGS

FIG. 1 schematically illustrates a method of treatment as described.

### DETAILED DESCRIPTION

In general, the compounds and methods described herein relate to medical foods for the treatment of developmentally-based neuropsychiatric disorders, and the formulation of these medical foods as well as the application or use of these medical foods to treat patients in need thereof. In particular, described herein are methods of determining that a patient would benefit from a medical food by analyzing one or more biomarkers. Also described herein are pharmaceutical compositions for treating developmentally-based neuropsychiatric disorders. In general these compositions (medical food or pharmaceutical compositions) include a methylglycine compound and an acetylcysteine compound.

As used herein the phrase “medical food” may refer to foods that are formulated and intended for the dietary management of a disease or disorder. These foods may provide distinctive nutritional elements that cannot be met by normal diet alone. Medical foods may be distinct from the broader category of foods for special dietary use and from traditional foods that bear a health claim. A medical food may be a food for oral ingestion or tube feeding (nasogastric tube), may be labeled for the dietary management of a specific medical disorder, disease or condition for which there are distinctive nutritional requirements, and may be intended to be used under medical supervision. Examples of medical foods may include: nutritionally complete formulas, nutritionally incomplete formulas, and formulas for metabolic disorders. Although the variations and examples described herein are specific to medical foods, in some variations the compositions described herein may be prepared and/or compounded as traditional “drugs” or medicaments.

The methods described herein are based on the determination to administer said composition utilizing a cluster of specific phenotypical and genotypical signals. These signals, herein described, include clinical and molecular aspects of perturbed brain development and include, but are not limited to, gene polymorphisms in modulatory systems involving the glutamate receptor (NMDAR) and nicotinic ACHr receptor, enzymes that regulate brain d-serine synthesis, oxidative pathways related to glutathione and neuregulin. Altered neuregulin (NRG1) in brain development, as a result of epigenetic and epistatic factors, is particularly relevant to the pathophysiology of schizophrenia and dysfunction of the NMDA receptor. NRG1 normally acts to promote NMDA activity via the phosphorylation of the NR2B subunit. Abnormal NRG1 signaling reduces NR2B and subsequently impairs NMDA and nACHR receptor function. Other genes (and polymorphisms) are also described.

Reductions in plasma and brain glycine, d-serine and glutathione levels, all of which provide potential mechanisms underlying NMDAR dysfunction. Thus, the NMDAR complex represents a convergence point for potential new treatment approaches in schizophrenia, and autism, which may involve general potentiation of pre- and post-synaptic glutamatergic and NMDAR function related to these disorders. NAC-Sarcosine complex enhance NR2B and thus may restore NRG1 mediated NMDA and nACHR functional impairments.

Biomarkers may be in the form of genes, proteins and other molecules, or phenotypical characteristics. Depending on the information they can provide, biomarkers may be used in diagnostics as prediction tools (e.g. subclinical markers, risk or vulnerability markers), or as diseases signatures (e.g. disease markers, stage or progression markers).

Although the pathophysiology of schizophrenia and autism remains unclear, there is an increasing body of evidence that several molecular pathways are involved. Neuroanatomical changes observed in psychotic disorders of childhood suggest an active biological process during the transition to full blown disease expression, raising the possibility that intervention might be indicated prior to expression of frank psychotic symptoms. Most findings point to the direction of malfunctioning of neurodevelopment signals and the glutamate pathway and the potential that oxidative stress is the cause of this disturbance.

An endophenotype may be neurophysiological, biochemical, endocrinological, neuroanatomical, cognitive, neuropsychological or genetic.

Autism and schizophrenia share common chromosomal susceptibility loci and many risk-promoting genes. Many genes associated with schizophrenia, autism and other psychotic disorders of childhood code for proteins associated with neurodevelopmentally related processes. These include NMDA and metabotropic glutamate receptors, growth factors (BDNF, NRG1), and many of their downstream signaling components (AKT1, DISC1, NOS1, Neuregulin), TNF, and CACNA1C, which mediates neuronal calcium signaling. The convergence of natural and genetic risk factors in autism and schizophrenia may help to explain the overlap in symptomatology.

Accruing data suggest that oxidative stress may be a critical factor underlying the pathophysiology of autism, and schizophrenia. Post-mortem prefrontal cortex from patients with each of these disorders have found that the levels of reduced, oxidized, and total Glutathione (GSH) were significantly decreased in all psychiatric conditions compared to the control groups. Results suggested an enhanced generation of reactive oxygen species and significantly lower free radical scavenging capacity in schizophrenia patients compared to healthy controls.

Indicators of oxidative stress are detectable in the urine. Significantly increased levels of isoprostanes were observed among schizophrenia patients relative to the controls, as measured by isoprostane-8-epi-prostaglandin F(2alpha) (8-isoPGF(2alpha)) concentrations in the urine. In further support that vulnerability to schizophrenia may be mediated by diminished brain antioxidant systems, microarray studies demonstrate up-regulation of SELENBP1 (selenium binding protein) in the brain and blood of patients with schizophrenia. Results demonstrate that SELENBP1 mRNA is upregulated in schizophrenic brains versus controls and, in addition, that SELENBP1 gene expression is strongly positively correlated with presence of psychosis across diagnoses. Furthermore, organic selenium compounds have been demonstrated to significantly reduce apomorphine-induced stereotyped behaviors in animals.

These lines of evidence point to the utility of raising antioxidant brain defense systems to mitigate the risk of developing a childhood psychotic disorder such as schizophrenia or autism. In particular, glutathione activity may be neuroprotective in these disorders by its influence on receptor interactions within receptor heterodimers and receptor mosaics, representing an important integrative mechanism for signaling based upon redox sensitive mechanisms in brain networks.

Current pathophysiological theories of schizophrenia emphasize that hypofunction of NMDA receptors at critical sites in local circuits modulate the function of a given brain region or control projections from one region to another (e.g., hippocampal-cortical or thalamocortical projections hypofunctional NMDA receptors such as glycine transporter inhibitors

N-methyl-D-aspartate (NMDA) receptors may play a critical role in the pathophysiology of schizophrenia, but the fundamental etiology of disturbed glutamate function remains unknown. The reduction in NMDA receptor function, as well as the reduced oxidative capacity etiologically associated with these disorders, are linked to impaired metabolic function of glycine in the brain. However, previous approaches to treat schizophrenia and autism with high doses of Glycine have been therapeutically unrewarding. Thus, the methods described herein have been developed to overcome these metabolic derangements through alternative pathways via the novel co administration of NAC and sarcosine.

Recognition that dissociative anesthetics block the N-methyl-D-aspartate (NMDA) receptor channel has inspired a search for glutamatergic therapeutic mechanisms because ketamine and phencyclidine are known to induce psychotic-like symptoms in healthy volunteers and exacerbate the symptoms of patients with schizophrenia. However, the mechanism whereby these agents disrupt glutamate signaling is particularly relevant to this invention.

Wistar rats treated with phencyclidine (10 mg/kg) exhibit region-specific changes characterized by decreased content of reduced glutathione (GSH). In hippocampus, reduced GSH content and decreased activities of GPx are induced by PCP administration. Furthermore, GSH-deficient mice displayed an increased locomotor response to low (2 and 3 mg/kg, i.p.) doses of phencyclidine. Moreover, the open field findings suggest reduced or altered N-methyl-d-aspartate (NMDA) receptor function in GSH-deficient mice.

Several lines of evidence also point to alterations of alpha-amino-3-hydroxyl-5-methyl-4-isoxazole-propionate (AMPA) receptor trafficking in schizophrenia. Alterations in AMPA receptor density after acute administration of PCP indicate that a reduced AMPA receptor activity may be a critical aspect in the disease. An overall decrease in levels of the glutamate AMPA receptor density in PCP treated rats has been demonstrated. More specifically, PCP-treated animals displayed decreased AMPA receptor density in hippocampus CA1 (−16%), hippocampus CA2 (−25%), and dentate gyrus (−27%). These studies support the notion that NMDA and AMPA receptor abnormalities are a consequence of reduced glutathione.

Modulation of glutamatergic transmission through distinct and selective receptor subtype mechanisms, such as potentiation of the N-methyl-D-aspartate (NMDA) receptor glycine site, activation of group II mGluR, and activation of glutamate-cystesine antiporters represent novel neurochemical targets to treat schizophrenia. Thus, the potential ability to positively modulate these receptors via the augmentation of brain glutathione by administration of a specific medical food represents a novel treatment. However, while it has been previously disclosed that the agents discussed in this discovery (N-acetylycysteine-Sarcosine), their implementation in preclinical stages of the disorder has not been previously disclosed. Further, the combination of these two medical foods provides a previously undisclosed synergy related to brain receptor function.

The tripeptide, glutathione (gamma-glutamylcysteinylglycine) is the primary endogenous free radical scavenger in the brain. When glutathione (GSH) levels are reduced there is increased cellular oxidative stress, characterized by an increase and accruement of reactive oxygen species (ROS). This may result in alterations in dopaminergic and glutamatergic activity implicated in these illnesses. Glutamate and dopamine are highly redox reactive molecules and produce free radicals during neurotransmission. Neurons are thus at high risk for oxidative injury and pro oxidative states have detrimental consequences on normal migrational processes and brain connectivity during development.

GSH is synthesised in two steps, catalyzed by two different enzymes. During the first step, gamma-glutamylcysteine synthetase (GCS) catalyses the formation of L-gamma-glutamyl-L-cysteine from glutamate and cysteine. The second step incorporates glycine under influence of glutathione synthetase, yielding GSH. GSH content is dependent on the supply of NAC, sarcosine and glycine. A major part of glycine is utilized for the synthesis of glutathione in astroglial cells

Synthesis of glutathione, a major redox regulator, is compromised in schizophrenia. The glutathione deficit, via its effect on redox-sensitive proteins could contribute to dysfunction of neurotransmitter systems in schizophrenia. Experimental models of glutathione deficit changed the modulation of responses by dopamine, from enhanced responses in control neurons (likely via D1-type receptors) to decreased responses in low-glutathione neurons (via D2-type receptors). This difference in dopamine modulation was due to a different modulation of L-type calcium channels activated during NMDA stimulation: dopamine enhanced function of these channels in control neurons but decreased it in low-glutathione neurons. The effect of a glutathione deficit on dopamine signaling was dependent on the redox-sensitive ryanodine receptors (RyRs), whose function was enhanced in low-glutathione neurons. This suggests that enhanced RyRs in low-glutathione neurons strengthens intracellular calcium-dependent pathways following activation of D2-type receptors and causes a decrease in function of L-type channels. This represents a mechanism by which dopaminergic systems could be dysfunctional under conditions of impaired glutathione synthesis as in schizophrenia. These changes closely mimic the pathological imbalances of dopamine signaling in schizophrenia, where D1 receptor function is blunted and D2 receptor activity is exaggerated.

Genetic studies have shown an association between schizophrenia and a GAG trinucleotide repeat (TNR) polymorphism in the catalytic subunit (GCLC) of the glutamate cysteine ligase (GCL), the key enzyme for glutathione (GSH) synthesis. This altered pattern potentially contributes to the development of a biomarker profile useful for early diagnosis and monitoring the effectiveness of novel treatments targeting redox dysregulation in schizophrenia.

Polymorphisms in the alpha(7) nicotinic acetylcholine receptor (nAChR) gene have been linked to schizophrenia. Genetic linkage studies implicated the alpha7 nAChRs subunit gene CHRNA7 in schizophrenia.

Nicotinic acetylcholine receptors (nAChRs) are membrane-bound, pentameric ligand-gated ion channels. Most known nAChRs contain an unusual eight-member disulfide-containing cysteinyl-cysteine ring. The cysteinyl-cysteine ring is located in a region implicated in ligand binding, and conformational changes involving this ring may be important for modulation of nAChR function.

Control of ligand-gated ion channel (LGIC) expression is essential for the formation, maintenance and plasticity of synapses. nACHR receptors may be down regulated by redox sensitive oxidative mechanisms resulting from disruption in the cysteinyl-cysteine disulfide ring. Thus, novel treatments which are directed at preserving the disulfide ring may prevent the pathological changes in the function of this receptor.

N-acetyl cysteine (NAC) is a precursor of cysteine and glutathione. It has antioxidant properties, lipid stabilization, and preservation of mitochondrial membrane potential, all of which may favorably impact receptor function in neuropsychiatric states. Treatment of neurons with lipid peroxidation byproducts results in a drastic reduction of mitochondrial membrane potential, and this reduction is prevented by NAC. This neuroprotective effect is due, at least in part, to preservation of mitochondrial membrane potential and intracellular GSH levels. Thus, NAC may exert neuroprotective effects via its ability to inhibit oxidation of mitochondrial proteins, and stabilization of receptor membrane dimers.

The carboxyl group in NAC is typically negatively charged at physiological pH, limiting its ability to cross cell membranes. N-acetylcysteine amide (NACA), a structural analogue of NAC, by replacing the carboxyl group with an amide, increases lipophilicity, allowing it to cross cell membranes and more readily crosses the blood-brain barrier. Thus, in some variations of the compositions and methods described herein, NACA is used in place of NAC.

NAC is also a potent glutamate modulator in the brain via its effects on the glutamate/cystine antiporter. The glutamate/cystine antiporter x(c)—transports cystine into cells in exchange for glutamate at a ratio of 1:1. Glutamate exported by system x(c)—is largely responsible for the extracellular glutamate concentration in the brain, whereas the imported cystine is required for the synthesis of the major endogenous antioxidant, glutathione. System x(c)—thus connects the antioxidant defense with neurotransmission and behavior. Disturbances in the function of system x(c)—have been implicated in nerve cell death due to increased extracellular glutamate and reduced intracellular glutathione. In vitro, inhibition of cystine import through system x(c)—leads to cell death by a mechanism called oxidative glutamate toxicity, which includes depletion of intracellular glutathione, activation of 12-lipoxygenase, accumulation of intracellular peroxides, and the activation of a cyclic guanosine monophosphate (cGMP)-dependent calcium channel towards the end of the death cascade. N-acetyl cysteine (NAC) inhibits glutamate via the cystine-glutamate exchange system. Further, by boosting glutathione, NAC acts as a potent antioxidant and has been shown in two positive, large-scale randomized placebo-controlled trials to affect negative symptoms in schizophrenia and depression in bipolar disorder.

N-acetylcysteine (NAC) treatment exerts its effects by activating cystine-glutamate exchange and thereby stimulating extrasynaptic metabotropic glutamate receptors (mGluR). NAC treatment of rats restored the ability to induce formation of new memories by indirectly stimulating mGluR2/3 and mGluR5, respectively. Thus, a previously undisclosed mechanism whereby NAC exerts beneficial effects in cognitive decline in pediatric neuropsychiatric disorders involves the facilitation of glutamate efflux and reduction of glutamate mediated excitotoxicity.

While the use of NAC has been proposed to be employed in clinical states of schizophrenia, its application and use in prodromal states and for the explicit purpose of preventing schizophrenia has not been previously disclosed (see, e.g., H H Chen, A Stoker, and A Markou, Psychopharmacology (Berl). 2010 May; 209(4):343-50). Further the particularly efficacious use of a combination of both NAC and sarcosine (N-methylglycine), which may produce results beyond what is separately achieved by either NAC or sarcosine alone, has not been previously described.

NMDARs are regulated in vivo by the amino acids glycine and D-serine. Sarcosine, a potent glycine transporter inhibitor, can increase synaptic glycine and promote NMDAR function.

Potentiation of the N-methyl-D: -aspartate (NMDA) receptor glycine site, activation of group II mGluR, and activation of glutamate-cysteine antiporters, are the therapeutic aspect of this invention. Medical food or Pharmacological manipulation of these specific NMDA receptor subtypes are recognized as being potentially as being efficacious in the treatment of schizophrenia and autism.

Sarcosine, also known as N-methylglycine, is an intermediate and byproduct in glycine synthesis and degradation Sarcosine is an amino acid involved in one-carbon metabolism and a promising therapy for schizophrenia, autism and other psychotic disorders characterized by impaired NMDA receptor function because it enhances NMDA receptor (NMDAR) function by inhibiting glycine uptake. Sarcosine is an NMDAR co-agonist at the glycine binding site.

Sarcosine is metabolized to glycine by the enzyme sarcosine dehydrogenase, while glycine methyl transferase generates sarcosine from glycine. Sarcosine is a natural amino acid and plays a significant role in various physiological processes and is the prime metabolic source glutathione. Sarcosine is a potent glycine transporter inhibitor and can increase synaptic glycine and promote NMDAR function. Sarcosine and N-acetylcysteine both ameliorated PPI deficits in mGluR5 knockout mice, pointing to their utility as treatments in schizophrenia.

The antipsychotic potential of sarcosine is supported by its ability to restore the prepulse inhibition (PPI) deficit, hyperlocomotion and regional brain c-Fos expression changes caused by an NMDAR antagonist, ketamine.

The combination of Sarcosine and N-acetylcysteine has not been previously described, and we herein predict an enhanced effect from this combination.

Treatment of Autism

As mentioned above, autism is a complex developmental disability that interferes with, among other things, the normal development of the brain in the areas of social interaction and communication skills. Although there is no known single known cause for autism, it is generally accepted that it is caused by abnormalities in brain structure or function. While researchers have not yet identified a single “trigger” that causes autism to develop, it also appears that some children are born with a susceptibility to autism. Among the candidate genes involved in the pathogenesis of autism, those particularly related to the effects of oxidative stress on brain development are most relevant to the invention.

Abnormal genes of oxidative stress pathways and increased oxidative stress have been reported in autism spectrum disorders. Polymorphisms of genes involved in glutathione metabolism, e.g. GSTP1, GSTM3 and GSTM1 are reportedly associated with autistic disorder. GPX1 GCG repeat and other gene polymorphisms such as the MnSOD ALA16 or the GPX1 Pro 198Leu polymorphism have also been reported in autism.

Furthermore, oxidized mitochondrial proteins are markedly increased in autism and altered Ca(2+) homeostasis play a key interactive role in the cascade of signaling events leading to autism. Plasma biomarkers of oxidative stress have been reported in autistic children and intracellular redox status GSH/GSSG redox ratio is decreased and percentage oxidized glutathione increased in both cytosol and mitochondria in the autism

Recent genetic studies have implicated a number of candidate genes in the pathogenesis of Autism Spectrum Disorder (ASD), which similar to schizophrenia, involve nACHr receptors, glutamate receptors, endogenous antioxidant pathways and altered calcium signaling. The overlap in these genes points to a convergence of abnormal brain development in both of these disorders.

Alpha4beta2 nAChRs and neurexin-1beta are coexpressed in hippocampal neurons, Interestingly, human neurexin-1 gene dysfunctions have been implicated in nicotine dependence and in autism spectrum disorders. Dysfunctional neurexins, through downstream effects on alpha4beta2, may contribute to the etiology of autism.

Alpha4 and beta2 protein expression and receptor binding density as well as alpha4 mRNA levels are lower in parietal cortex in autism, while alpha7 did not change for any of these parameters. The data obtained, using complementary measures of receptor expression, indicate that reduced gene expression of the alpha4beta2 nicotinic receptor in the cerebral cortex is a major feature of the neurochemical pathology of autism, whilst post-transcriptional abnormalities of both this and the alpha7 subtype are apparent in the cerebellum. The findings point to dendritic and/or synaptic nicotinic receptor abnormalities that may relate to disruptions in cerebral circuitry development.

Based on 1) neuroanatomical and neuroimaging studies indicating aberrations in brain regions that are rich in glutamate neurons and 2) similarities between symptoms produced by N-methyl-D-aspartate (NMDA) antagonists in healthy subjects and those seen in autism, it has been proposed that autism may be related to hypoglutamatergic function. The possible benefit of treatment with glutamate agonists [e.g. agents acting on the modulatory glycine site of the NMDA receptor, or so-called ampakines acting on the alpha-amino-3-hydroxy-5-methyl-4-isoxazole propionic acid (AMPA) receptor] have been considered for the treatment of autism.

Plasma levels of metabolites in methionine transmethylation and transsulfuration pathways were measured in 80 autistic and 73 control children. The metabolic results indicated that plasma methionine and the ratio of S-adenosylmethionine (SAM) to S-adenosylhomocysteine (SAH), an indicator of methylation capacity, were significantly decreased in the autistic children relative to age-matched controls. In addition, plasma levels of cysteine, glutathione, and the ratio of reduced to oxidized glutathione, an indication of antioxidant capacity and redox homeostasis, were significantly decreased. Differences in allele frequency and/or significant gene-gene interactions were found in autistic individuals. Further, methylenetetrahydrofolate reductase (MTHFR 677C>T and glutathione-S-transferase (GST M1) display polymorphisms in autism.

Cytosolic serine hydroxyl methyl transferase (SHMT1 C1420T) allele frequency was found to be abnormal in autistic children compared with nonautistic children (16.3 vs. 6.5%) with 2.79-fold increased risk for autism [95% confidence interval (CI): 1.58-4.93]. The SHMT 1420T allele was lower in autistic group compared with nonautistic group, indicating a metabolic disturbance of folate/serine/sarcosine pathways in the etiopathogenesis of autism.

Oxidative stress in autism has been studied at the membrane level and also by measuring products of lipid peroxidation, detoxifying agents (such as glutathione), and antioxidants involved in the defense system against reactive oxygen species (ROS). Lipid peroxidation markers are elevated in autism, indicating that oxidative stress is increased in this disease. Levels of major antioxidant serum proteins, namely transferrin (iron-binding protein) and ceruloplasmin (copper-binding protein), are decreased in children with autism. There is a positive correlation between reduced levels of these proteins and loss of previously acquired language skills in children with autism.

The membrane phospholipids, the prime target of ROS, are also altered in autism. The levels of phosphatidylethanolamine (PE) are decreased, and phosphatidylserine (PS) levels are increased in the erythrocyte membrane of children with autism as compared to their unaffected siblings. Several studies have suggested alterations in the activities of antioxidant enzymes such as superoxide dismutase, glutathione peroxidase, and catalase in autism. Additionally, altered glutathione levels and homocysteine/methionine metabolism, increased inflammation, excitotoxicity, as well as mitochondrial and immune dysfunction have been suggested in autism

Taken together, these studies suggest increased oxidative stress in autism that may contribute to the development of this disease. A mechanism linking oxidative stress with membrane lipid abnormalities, inflammation, aberrant immune response, impaired energy metabolism and excitotoxicity, leading to clinical symptoms and pathogenesis of autism suggests that interventions which restore anti oxidant defense systems may reduce the vulnerability to the expression of this disorder. Thus, a previously undisclosed invention relates to the administration of a medical food product comprising the combination of N acetylcysteine and sarcosine in order to raise brain glutathione levels. While it has been previously disclosed that the use of these agents may benefit autistic symptomatology, the invention disclosed herein suggests both the unique and synergestic combination of these two agents as well as their administration in preclinical stages of the disorder.

The metabolic fate of glycine in the brain plays a critical role in the pathogenesis of schizophrenia and autism. Glycine serves as a precursor for serine, which in turn acts as a co agonist of NMDA receptors and a precursor for glutathione synthesis. However, previous attempts to ameliorate these abnormalities via the administration of glycine or serine have been disappointing. This is because of a previously unrecognized metabolic defect in serine function which may be overcome by the co administration of NAC and sarcosine.

An important element of the discovery relates to the critical importance of maintaining adequate blood levels of the medical food. Sarcosine and NAC have short half lives

Normal subjects clear sarcosine from plasma very rapidly (t(½), 1.6 hr). After an oral dose of N-acetylcysteine 200 to 400 mg has a terminal half-life of 6.25 h. Thus, to achieve a therapeutic response in autism or schizophrenia, an individual may require frequent dosing. Therefore, an improvement in the application of these compounds may involve a controlled delivery mechanism that would ensure continuous blood levels to achieve a desired therapeutic response.

Dissolution-controlled methods are claimed as a preferential means to administer the sarcosine-N acetylcysteine formulation.

In these products, the rate of dissolution of the compound (and thereby availability for absorption) is controlled by slowly soluble polymers or by microencapsulation. Once the coating is dissolved, the drug becomes available for dissolution. By varying the thicknesses of the coat and its composition, the rate of drug release can be controlled.

The release of drug from these products is controlled by the erosion rate of a carrier matrix. The rate of release is determined by the rate of erosion. An ideal carrier agent in this regard may be choline. Choline administration may further the efficacy of this compound by acting directly as a nicotinic ACHr receptor agonist.

Other methods of sustained delivery of the compound, known to those skilled in the art, are also claimed. The present invention relates to a method of administration and a medical food or pharmaceutical composition containing N-acetylcysteine and sarcosine as the active ingredient which provide increased levels of unmodified drug in the blood following oral administration.

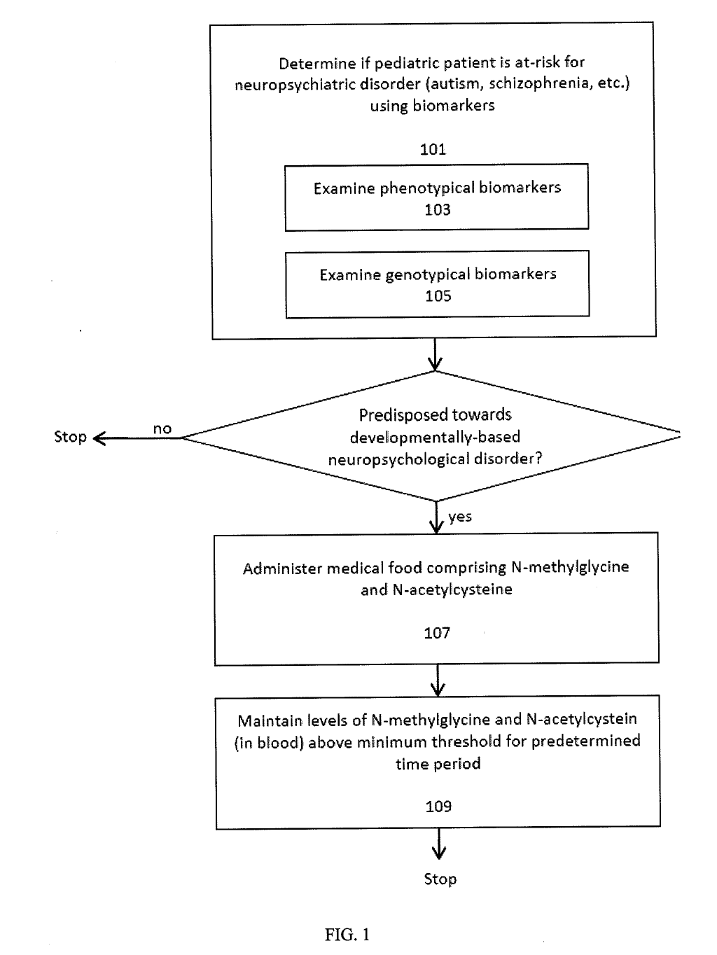

FIG. 1 illustrates one exemplary method of treating a patient. For example, in FIG. 1, the first step 101 includes the identification of children at risk for autism or schizophrenia and other childhood psychotic disorders. This may be achieved via the utilization of biomarkers, as just described. For example, these biomarkers may include endophenotypes, identification of particular symptomatology (for instance, subclinical psychotic symptoms including transient psychosis, disorganization or in autism-delayed language, regression of milestones, stereotypical motor behaviors, repetitive movements); combined with biomarkers which reveal increased oxidative stress and/or genetic markers as described in the paragraphs above.

Next, 103, in patients in which the biomarkers indicate an enhanced risk or susceptibility of developing the disorder, the patient may be prescribed an oral administration of a medical food composition comprising NAC-Sarcosine, or the salts thereof, as described above. The medical food or pharmacological agent is typically delivered so as to maintain and ensure sustained elevated blood levels on a continuous basis to improve the function of children with subthreshold manifestations of psychosis or autism. Thus, the level may be sustained for a predetermined time period (e.g., 4 hours, 8 hours, 12 hours, 24 hours, etc.) and repeated administration may allow for more prolonged sustained elevation.

In some variations of the compositions described herein, the compositions may include between about 100 mg of each component to 2000 mg each component. For example, in some variations, the compositions may include a dose range of about 500 to about 1000 mg NAC-Sarcosine per dose.

While the compositions, methods of forming them, and methods for using them, have been described in some detail here by way of illustration and example, such illustration and example is for purposes of clarity of understanding only. It will be readily apparent to those of ordinary skill in the art in light of the teachings herein that certain changes and modifications may be made thereto without departing from the spirit and scope of the invention.

What is claimed is:

1. A method of treating a developmentally-based neuropsychiatric disorder comprising:
administering a medical food composition comprising a therapeutically effective amount of a first compound and a second compound, wherein the first compound is an N-methylglycine compound;
further wherein the second compound is an acetylcysteine compound;
wherein the medical food composition is administered at a dose equivalent to 10 mg to 10 g per day of the first compound and 100 mg-10 g per day of the second compound.

2. The method of claim 1, wherein the first compound is one of: N-methylglycine, a salt of N-methylglycine, or an ester of N-methylglycine.

3. The method of claim 1, wherein the first compound is a precursor of N-methylglycine.

4. The method of claim 1, wherein the first compound is N,N,N-trimethylglycine or N,N-dimethylglycine.

5. The method of claim 1, where the second compound is the amide salt of N-acetylcysteine.

6. The method of claim 1, wherein the neuropsychiatric disorder is schizophrenia.

7. The method of claim 1, wherein the neuropsychiatric disorder is autism.

8. The method of claim 1, wherein the neuropsychiatric disorder is pervasive developmental delay.

9. The method of claim 1 wherein the neuropsychiatric disorder is childhood psychotic disorder.

10. The method of claim 1, further comprising determining if a patient is suffering from a neuropsychiatric disorder.

11. The method of claim 1, further comprising determining if a patient is at risk from a neuropsychiatric disorder using a phenotypical and genotypical biomarker when the patient is in preclinical or prodromal stages of a pediatric neuropsychiatric disorder.

12. The method of claim 1, wherein the step of administering comprises maintaining a sustained elevated blood level of the first and second compound on a continuous basis.

13. A pharmaceutical composition for the treatment of developmentally-based neuropsychiatric disorders, the composition comprising:
a first compound, wherein the first compound is an N-methylglycine compound at a concentration of about 10 mg to 10 g;
a second compound, wherein the second compound is an acetylcysteine compound at a concentration of about 100 mg-10 g.

14. The composition of claim 13, wherein the first compound is selected from the group consisting of: N-methylglycine, a salt of N-methylglycine, or an ester of N-methylglycine, a precursor of N-methylglycine, N,N,N-trimethylglycine or N,N-dimethylglycine.

15. The composition of claim 13, wherein the second compound is the amide salt of N-acetylcysteine.

16. The composition of claim 13, wherein the first composition is compounded for the sustained release of at least 90% of the first and second compounds over a 12 hour period.

17. The composition of claim 13, wherein the first composition is compounded for the sustained release of at least 90% of the first and second compounds over a 24 hour period.

18. The composition of claim 13, wherein the concentration of both the first and second compounds is between about 100 mg and about 2 g.

19. The composition of claim 13, wherein the concentration of both the first and second compounds is between about 500 mg and about 1 g.

20. A medical food composition comprising:
a therapeutically effective amount of a first compound, wherein the first compound is an N-methylglycine compound at a dose equivalent of about 10 mg to 10 g per day;
a therapeutically effective amount of a second compound, wherein the second compound is an acetylcysteine compound at a dose equivalent of about 100 mg-10 g per day.

21. The medical food composition of claim 20, wherein the first compound is selected from the group consisting of: N-methylglycine, a salt of N-methylglycine, or an ester of N-methylglycine, a precursor of N-methylglycine, N,N,N-trimethylglycine or N,N-dimethylglycine.

22. The medical food composition of claim 20, wherein the second compound is the amide salt of N-acetylcysteine.

23. The medical food composition of claim 20, wherein the concentration of both the first and second compounds is between about 100 mg and about 2 g.

24. The medical food composition of claim 20, wherein the concentration of both the first and second compounds is between about 500 mg and about 1 g.
